(19) United States
(12) Patent Application Publication
(10) Pub. No.: US 2008/0097110 A1
(43) Pub. Date: Apr. 24, 2008

(54) Salt forms of substituted benzothienyl compounds

(76) Inventors: Luigi Anzalone, West Chester, PA (US); Frank John Villani, Perkasie, PA (US); Penina Feibush, Ambler, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/975,571

(22) Filed: Oct. 19, 2007

Related U.S. Application Data
(60) Provisional application No. 60/853,407, filed on Oct. 20, 2006.

(51) Int. Cl. C07D 333/50

(52) U.S. Cl. 549/6

(57) ABSTRACT

The present invention relates to novel salt forms of a compound of Formula (I): 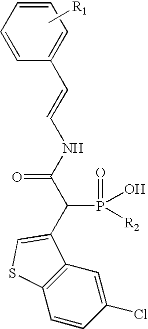 and processes for their preparation.

### CROSS REFERENCE TO RELATED APPLICATIONS

This present application claims benefit of U.S. Provisional Patent Application Ser. No. 60/853,407 filed Oct. 20, 2006, which is incorporated herein by reference in its entirety and for all purposes.

### FIELD OF THE INVENTION

The present invention relates to novel salt forms of a series of substituted benzothienyl compounds and processes for their preparation.

### BACKGROUND OF THE INVENTION

U.S. Patent Application Publication No. 2005/0176769 (published Aug. 11, 2005) discloses a class of compounds, and novel salt forms thereof, which selectively inhibit binding to the chymase receptor.

### SUMMARY OF THE INVENTION

The present invention is directed to substituted benzothienyl compounds of Formula (I): 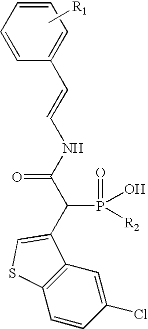
and novel salt forms thereof, wherein R₁ and R₂ are as defined herein.

It is desirable to have the compound of Formula (I) present as a salt form. Salt forms are generally more soluble in water, more bioavailable and are easier to handle in the production of tablets and other dosage formulations.

The present invention is also directed to salt forms of the compound of Formula (I), such as a benzathine, t-butylamine, magnesium, calcium, choline, cyclohexylamine, diethanolamine, ethylenediamine, L-lysine, NH₃, NH₄OH, N-methyl-D-glucamine, piperidine, potassium, procaine, quinine, sodium, triethanolamine, imidazole or tris(hydroxymethyl)methylamine (tromethamine) salt.

The present invention is further directed to a process for the preparation of said salt forms of the compound of Formula (I).

### DETAILED DESCRIPTION OF THE INVENTION

The present invention is directed to salt forms of substituted benzothienyl compounds of Formula (I): 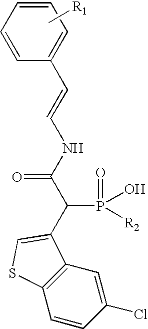
wherein
R₁ is one or two halogen substituents; and,
R₂ is C₁₋₄alkyl, C₁₋₄alkoxy, pivalyloxy-C₁₋₄alkoxy or hydroxy.

An example of the present invention includes salt forms of a compound of Formula (I) wherein
R₁ is two halogen substituents, wherein halogen is selected from fluoro or chloro; and,
R₂ is C₁₋₄alkyl, pivalyloxy-C₁₋₄alkoxy or hydroxy.

Examples of the present invention include a salt of a compound of Formula (I) selected from:

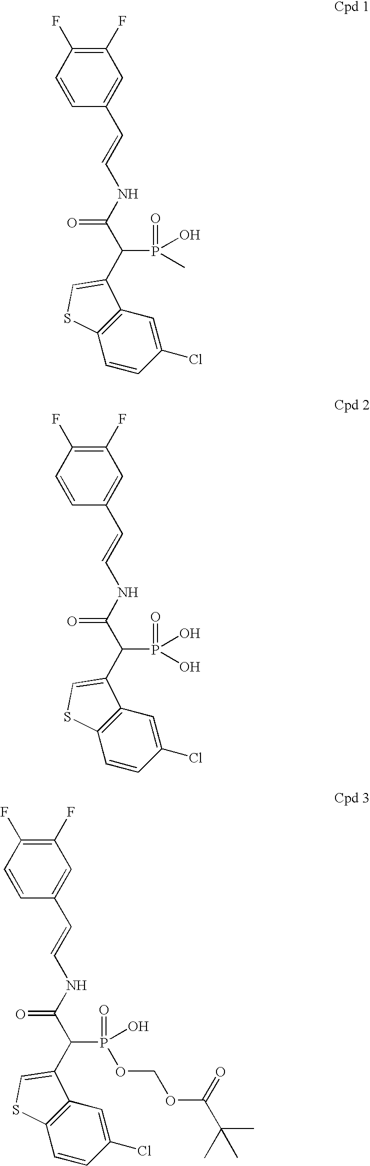

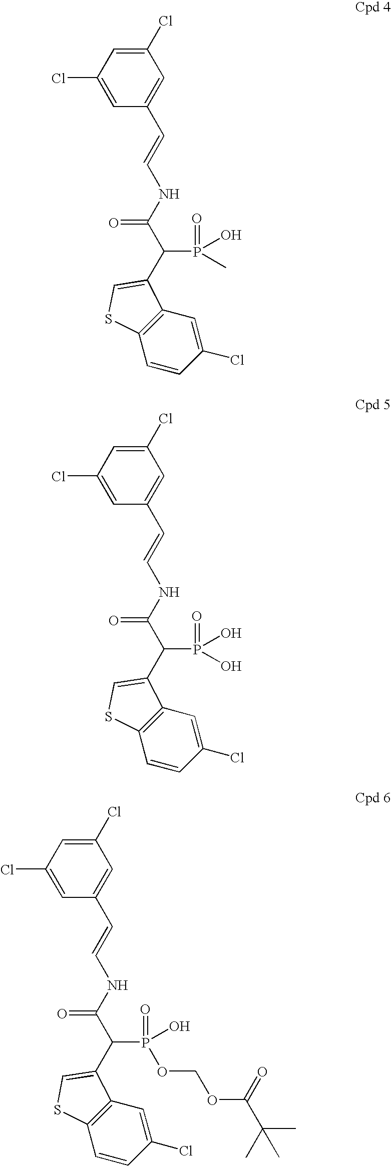

In U.S. Patent Application Publication 2005/0176769: {(5-chloro-benzo[b]thiophen-3-yl)-[2-(3,4-difluoro-phenyl)-vinylcarbamoyl]-methyl}-methyl-phosphinic acid (Compound 1, above) was disclosed as Compound 17 and the preparation of the free acid and tromethane salt was described in Example 6; {(5-chloro-benzo[b]thiophen-3-yl)-[2-(3,4-difluoro-phenyl)-vinylcarbamoyl]-methyl}-methyl-phosphonic acid (Compound 2, above) was disclosed as Compound 2 and the preparation of the free acid described in Example 11; 2,2-dimethyl-propionic acid {(5-chloro-benzo[b]thiophen-3-yl)-[2-(3,4-difluoro-phenyl)-vinylcarbamoyl]-methyl}-hydroxy-phosphinoyloxymethyl ester (Compound 3, above) was disclosed as Compound 187 and the preparation of the free acid was described in Example 51; {(5-chloro-benzo[b]thiophen-3-yl)-[2-(3,5-dichloro-phenyl)-vinylcarbamoyl]-methyl}-methyl-phosphinic acid (Compound 4, above) was disclosed as Compound 170 and the free acid was prepared using the procedure of Example 6; {(5-chloro-benzo[b]thiophen-3-yl)-[2-(3,5-dichloro-phenyl)-vinylcarbamoyl]-methyl}-phosphonic acid (Compound 5, above) was disclosed as Compound 207 and the free acid may be prepared using the procedure of Example 11; and, 2,2-dimethyl-propionic acid {(5-chloro-benzo[b]thiophen-3-yl)-[2-(3,5-dichloro-phenyl)-vinylcarbamoyl]-methyl}-hydroxy-phosphinoyloxymethyl ester (Compound 6, above) was disclosed as Compound 261 and the free acid may be prepared using the procedures of Example 51 and Example 11.

The present invention is also directed to salt forms of the compound of Formula (I), such as a benzathine, t-butylamine, magnesium, calcium, choline, cyclohexylamine, diethanolamine, ethylenediamine, L-lysine, NH₃, NH₄OH, N-methyl-D-glucamine, piperidine, potassium, procaine, quinine, sodium, triethanolamine, imidazole or tris(hydroxymethyl)methylamine salt.

Embodiments of the present invention include salts such as a mono-benzathine, mono-t-butylamine, mono-magnesium, mono-calcium, mono-choline, mono-cyclohexylamine, mono-diethanolamine, mono-ethylenediamine, mono-L-lysine, mono-NH₃, mono-NH₄OH, mono-N-methyl-D-glucamine, mono-piperidine, mono-potassium, mono-procaine, mono-quinine, mono-sodium, mono-triethanolamine, mono-imidazole or mono-tris(hydroxymethyl)methylamine salt of the compound of Formula (I).

Embodiments of the present invention include salts such as a mono-magnesium, mono-calcium, mono-choline, mono-N-methyl-D-glucamine, mono-potassium, mono-sodium or mono-tris(hydroxymethyl)methylamine salt of the compound of Formula (I).

Embodiments of the present invention include crystalline forms of the mono-benzathine, mono-t-butylamine, mono-magnesium, mono-calcium, mono-choline, mono-cyclohexylamine, mono-diethanolamine, mono-ethylenediamine, mono-L-lysine, mono-NH₃, mono-NH₄OH, mono-N-methyl-D-glucamine, mono-piperidine, mono-potassium, mono-procaine, mono-quinine, mono-sodium, mono-triethanolamine, mono-imidazole or mono-tris(hydroxymethyl)methylamine (tromethamine) salts of the compound of Formula (I).

Examples of the present invention include crystalline forms of the mono-magnesium, mono-calcium, mono-choline, mono-N-methyl-D-glucamine, mono-potassium, mono-sodium or mono-tris(hydroxymethyl)methylamine salts of the compound of Formula (I).

Embodiments of the present invention include the mono-choline salt as an anhydrous or di-hydrate form.

Embodiments of the present invention include the mono-choline or mono-N-methyl-D-glucamine salt as an unsolvated form, a solvated form or an amorphous form.

Embodiments of the present invention include the mono-choline salt as an unsolvated form, a solvated form or an amorphous form.

Embodiments of the present invention include the mono-choline salt of a compound selected from the group consisting of:
{(5-chloro-benzo[b]thiophen-3-yl)-[2-(3,4-difluoro-phenyl)-vinylcarbamoyl]-methyl}-methyl-phosphinic acid,
{(5-chloro-benzo[b]thiophen-3-yl)-[2-(3,4-difluoro-phenyl)-vinylcarbamoyl]-methyl}-phosphonic acid,
2,2-dimethyl-propionic acid {(5-chloro-benzo[b]thiophen-3-yl)-[2-(3,4-difluoro-phenyl)-vinylcarbamoyl]-methyl}-hydroxy-phosphinoyloxymethyl ester,
{(5-chloro-benzo[b]thiophen-3-yl)-[2-(3,5-dichloro-phenyl)-vinylcarbamoyl]-methyl}-methyl-phosphinic acid,
{(5-chloro-benzo[b]thiophen-3-yl)-[2-(3,5-dichloro-phenyl)-vinylcarbamoyl]-methyl}-phosphonic acid, and
2,2-dimethyl-propionic acid {(5-chloro-benzo[b]thiophen-3-yl)-[2-(3,5-dichloro-phenyl)-vinylcarbamoyl]-methyl}-hydroxy-phosphinoyloxymethyl ester.

An embodiment of the present invention is the mono-choline salt of {(5-chloro-benzo[b]thiophen-3-yl)-[2-(3,4-difluoro-phenyl)-vinylcarbamoyl]-methyl}-methyl-phosphinic acid.

The present invention is further directed to a process for the preparation of said salt forms of the compound of Formula (I).

The preparation of the salt forms of the compound of Formula (I) is generally described in Scheme A.

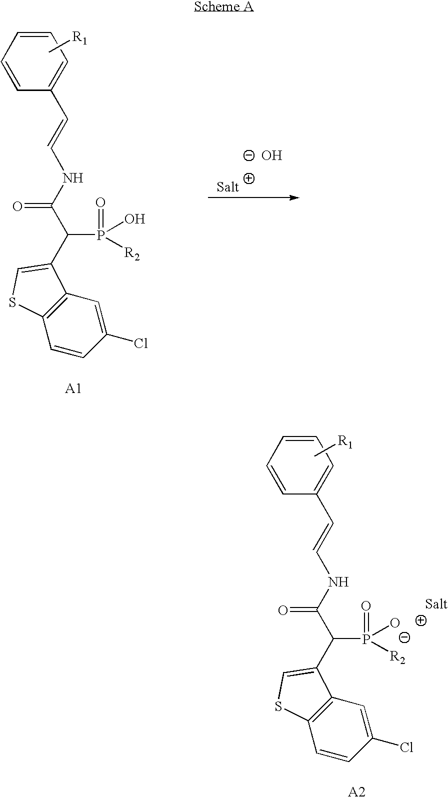

One equivalent of a solvated free acid form of a Compound A1 (in a solvent such as methanol, ethanol and the like) in a suitable additional amount of a first solvent (such as methanol, ethanol and the like) is prepared in a round-bottomed flask equipped with a mechanical stirrer, an addition funnel and a distillation condenser under an inert atmosphere (using a gas such as nitrogen). The resulting mixture is reacted with an equivalent of a solvated salt (in a polar organic solvent such as methanol, ethanol, ethyl acetate, isopropyl alcohol, and the like or mixtures thereof), then the reaction product is aged and filtered. The first solvent is removed by distillation and a second solvent (such as ethyl acetate) is added. The mixture is seeded with crystals of the salt and then worked up to provide a salt form of a Compound A2.

The equivalent of the salt used in Scheme A for reaction with Compound A1 is in a range of from about 0.96 to about 1.16 molar equivalents, a range of from about 0.99 to about 1.13 molar equivalents, a range of from about 1.02 to about 1.1 molar equivalents, or a range of from about 1.04 to about 1.08 molar equivalents.

The reaction of an equivalent of a solvated salt with the solution of a free acid mixture described in Scheme A may be carried out using a salt that is in the form of either a solid or a gas and includes, without limitation, salts in a form which are known to those skilled in the art for use as described herein.

The solvents described for use in Scheme A are for illustrative purposes only and include, without limitation, those which are known to those skilled in the art for use as described herein but are preferably anhydrous.

The means of work up for obtaining the salt form of a Compound A2 referred to in Scheme A includes, without limitation, precipitating the salt by seeding the salt mixture in a solvent with crystals of the salt form, precipitating the salt by cooling, use of an antisolvent or by vapor diffusion crystallization with an antisolvent, forming the salt by rapid evaporation of the solvent from the salt mixture, preparing and quenching a melt of the salt form (for example by pouring the melt onto a cold plate), heating a salt form to a suitable temperature and allowing the sample to cool at room temperature, slowly evaporating the solvent from the salt mixture (for example, by allowing the solvent to evaporate under room temperature),

When recovering the salt by crystallization with an anti-solvent, suitable solvent:antisolvent pairs include methanol:acetone, water:acetone, ethanol:ethyl acetate and methanol:ethyl acetate.

When recovering the salt by vapor diffusion crystallization with an anti-solvent, suitable solvent:antisolvent pairs include dichloromethane:acetone, dichloromethane:diethyl ether, dichloromethane:hexanes, dichloromethane:tetrahydrofuran and N,N-dimethylformamide:toluene.

### DEFINITIONS

The term “mono” salt of the compound of Formula (I) means a salt form of the compound of Formula (I) wherein the molar ratio of the compound of Formula (I) to the salt ion is 1:1.

The abbreviation “KF” means the weight percent of water in a product, as determined by the Karl-Fischer test.

The term “anti-solvent” means a solvent which does not dissolve a specific substance and is added to a solution of the substance, directly or by vapor diffusion, to cause precipitation of said substance.

The term “C₁₋₄alkyl” whether used alone or as part of a substituent group refers to straight and branched carbon chains having 1 to 4 carbon atoms or any number within this range. Examples include methyl, ethyl, 1-propyl, 2-propyl, 1-butyl, 2-butyl, tert-butyl and the like.

The term “C₁₋₄alkoxy” refers to a substituent of the formula: —O-alkyl substituent group. Examples include methoxy, ethoxy, propoxy and the like.

The term “pivaloyloxy-C₁₋₄alkoxy” refers to a substituent of the formula: —O—C₁₋₄alkyl-O—C(CH₃)₃.

The term “halogen” refers to fluorine, chlorine, bromine and iodine.

The following examples describe the invention in greater detail and are intended to illustrate the invention, but not to limit it.

### EXAMPLE 1

### {(5-chloro-benzo[b]thiophen-3-yl)-[2-(3,4-difluoro-phenyl)-vinylcarbamoyl]-methyl}-methyl-phosphinic Acid Choline (Compound 1a)

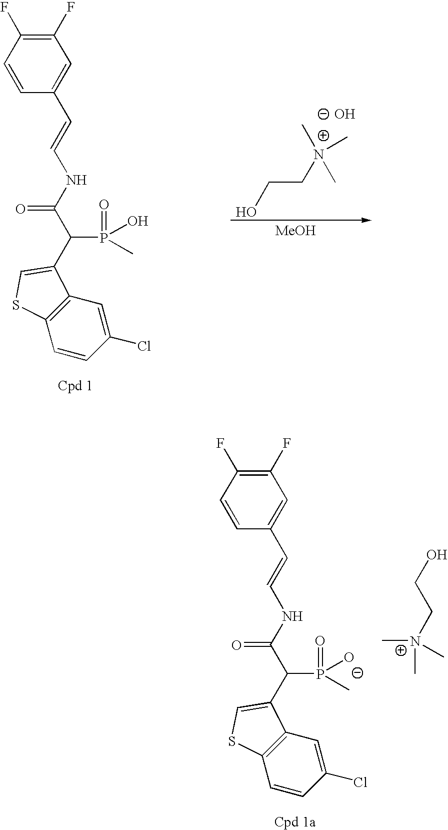

To a 5.0 L 4-necked round-bottomed flask equipped with a mechanical stirrer, an addition funnel, and a distillation condenser, was added Compound 1 (393 g, 0.83 mol, 1 eq) of free acid (methanol solvated) and 3.4 L of methanol under N₂. The resulting slurry was treated with (237 g, 0.88 mol, 1.06 eq) of 45% by wt. of choline hydroxide in methanol added at once (slight exotherm, from 16° C. to 20° C.). A homogeneous solution was obtained shortly after the addition. The solution was aged at room temperature for about 1 hr and then clarified by filtration through a sintered glass (medium).

A quantity of 2.0 L of methanol was removed by distillation (about 63° C.) and replaced with 2.0 L of ethyl acetate, added slowly over a period of about 15 min. to about 30 min. to maintain the temperature. The clear solution was seeded with crystals of choline salt, and then cooled slowly to RT under moderate agitation. Precipitation commenced during this period. The resulting slurry was aged at ambient temperature over-night and then filtered. The solid was washed with 80 ml of cold EtOAc and dried in a 60° C. vac-oven (O/N) to yield Compound 1a (18.2 g, 79.1%) of white solid. KF: 0.18%; DSC melting point onset/peak maximum: 249.1° C./252.0° C.

Using the procedure of Example 1, and various other starting materials, reagents and solvents and conditions known to those skilled in the art, the following salt forms may be prepared:

| Cpd | Name |
|---|---|
| 2 | {(5-chloro-benzo[b]thiophen-3-yl)-[2-(3,4-difluoro-phenyl)- |
| | vinylcarbamoyl]-methyl}-phosphonic acid choline |
| 3 | 2,2-dimethyl-propionic acid{(5-chloro-benzo[b]thiophen-3-yl)-[2- |
| | (3,4-difluoro-phenyl)-vinylcarbamoyl]-methyl}-hydroxy- |
| | phosphinoyloxymethyl ester choline |
| 4 | {(5-chloro-benzo[b]thiophen-3-yl)-[2-(3,5-dichloro-phenyl)- |
| | vinylcarbamoyl]-methyl}-methyl-phosphinic acid choline |
| 5 | {(5-chloro-benzo[b]thiophen-3-yl)-[2-(3,5-dichloro-phenyl)- |
| | vinylcarbamoyl]-methyl}-phosphonic acid choline |
| 6 | 2,2-dimethyl-propionic acid {(5-chloro-benzo[b]thiophen-3-yl)-[2- |
| | (3,5-dichloro-phenyl)-vinylcarbamoyl]-methyl}-hydroxy- |
| | phosphinoyloxymethyl ester choline |

The salt forms of Compound 1 may be characterized by an X-ray diffraction pattern (pXRD).

The pXRD pattern for Compound 1 is listed in Table 1 and was backloaded into a conventional x-ray holder and analyzed as received using the X-Celerator detector. The sample was scanned from 3 to 35 °2θ at a step size of 0.0165 °2θ and a time per step of 10.16 seconds. The effective scan speed is 0.20670/s. Instrument voltage and current settings of 45 kV and 40 mA were employed.

The crystalline choline salt of Compound 1 was characterized by pXRD, wherein position is shown as °2θ, d-spacing is shown as Å and percent relative intensity is shown as %, comprising the peaks:

TABLE 1

| °2θ | Å | % |
|---|---|---|
| 8.328 | 10.6177 | 71.32 |
| 10.069 | 8.7850 | 13.18 |
| 12.064 | 7.3367 | 12.10 |
| 14.202 | 6.2364 | 77.83 |
| 16.382 | 5.4110 | 34.77 |
| 18.599 | 4.7708 | 10.95 |
| 19.206 | 4.6213 | 100.00 |
| 19.845 | 4.4740 | 53.54 |
| 19.955 | 4.4496 | 57.38 |
| 20.181 | 4.4002 | 60.89 |
| 20.584 | 4.3151 | 40.36 |
| 21.101 | 4.2104 | 11.74 |
| 21.300 | 4.1715 | 13.23 |
| 22.089 | 4.0243 | 83.79 |
| 22.833 | 3.8949 | 50.10 |
| 24.049 | 3.7006 | 26.09 |
| 25.257 | 3.5262 | 14.17 |
| 25.894 | 3.4409 | 11.04 |
| 26.713 | 3.3373 | 30.15 |
| 28.522 | 3.1296 | 36.20 |
| 29.733 | 3.0048 | 18.27 |
| 30.521 | 2.9266 | 19.90 |
| 31.579 | 2.8333 | 14.15 |

### EXAMPLE 2

Using the procedure of Example 1, a second choline salt form Compound 1b was recrystallized from EtOH/MTBE. Trace solvent: 2.50% EtOH, 1.7% MTBE; DSC melting point onset/peak maximum:

244.5° C./248.3° C.

A comparison of the Compound 1a pXRD and Compound 1b pXRD showed an enhanced resolution of features in Compound 1a relative to Compound 1b under the same pXRD conditions. This supports the finding that the trace solvent profile was lower for Compound 1a than for Compound 1b.

### EXAMPLE 3

Using the procedure of Example 1 and other conventional methods known to those skilled in the art, additional salt forms representative of the present invention were prepared and characterized as shown in Table 2. The Differential Scanning Calorimetry melting point (M.P.) is shown at onset and peak maximum as onset/peak max.

TABLE 2

| Form | | KF | Trace Solvent | M.P. (° C.) | Form |
|---|---|---|---|---|---|
| A | free acid | 0.34% | 6.33% MeOH | 102.3/117.8 | crystalline |
| | | | | | methanolate |
| B1 | tromethane | 0.17% | 0.24% EtOH, | | crystalline |
| | salt | | 0.82% MTBE | | |
| B2 | tromethane | | | 185/188 | crystalline |
| | salt | | | | |
| C1 | N-methyl | 2.19% | 2.50% EtOH, | | partially crystalline |
| | glucamine salt | | 2.0% MTBE | | |
| C2 | recrystallized | | | 115/120 | partially crystalline |
| | Form D | | | | |
| D | calcium salt | 5.25% | none | | partially crystalline |
| E | sodium salt | | | | amorphous |
| F1 | potassium salt | | | | crystalline |
| F2 | potassium salt | | | | amorphous |
| F3 | potassium salt | | | | partially crystalline |
| H | magnesium | | | | amorphous |
| | salt | | | | |

### EXAMPLE 4

Dynamic vapor sorption (DVS) testing was performed on several salt forms and sorption and desorption results under various relative humidity (RH) conditions are shown in Table 3. Results indicated that the choline salt was the least hygroscopic of the crystalline forms.

TABLE 3

| Form | Sorption | Desorption |
|---|---|---|
| A | 0-30% RH: 0.34% | 90-30% RH: 1.48% |
| | 30-90% RH: 2.60% | 30-0% RH: 7.14% |
| | 0-90% RH: 2.94% | |
| Compound 1b | 0-90% RH: 1.308% | 90-0% RH: 1.101% |
| B2 | 0-90% RH: 2.085% | 90-0% RH: 2.231% |
| C2 | 0-80% RH: 3.68% | 90-10% RH: 3.91% |
| | 80-90% RH: 1.84% | 10-0% RH: 2.75% |
| | 0-90% RH: 5.52% | 90-0% RH: 6.66% |
| D | 0-90% RH: 4.62% | 90-0% RH: 6.18% |
| F1, F2, F3 | 0-60% RH: 0.00% | 90-0% RH: 16.81% |
| | 60-70% RH: 0.30% | |
| | 70-80% RH: 2.46% | |
| | 80-90% RH: 8.59% | |
| | 0-90% RH: 11.35% | |

### EXAMPLE 5

Solubility testing was performed on several salt forms and results for free compound (mg) in media (ml) (represented as mg/ml) at equilibrium solubility are shown in Table 4. SIF refers to simulated intestinal fluid.

The equilibrium solubility could not be determined for N-methyl-D-glucamine Form C2, which remained in solution at 232 mg/ml.

TABLE 4

| Form | Media | pH | mg/ml |
|---|---|---|---|
| A | 0.1N HCL | 1.50 | 5.7 |
| | SIF | 4.78 | 18.7 |
| | 0.1N NaOH | 4.47 | 46.5 |
| Compound 1b | 0.1N HCL | 1.40 | 6.0 |
| | SIF | 7.85 | 21.5 |
| | 0.1N NaOH | 11.78 | 45.2 |
| B1 | 0.1N HCL | 1.51 | 1.7 |
| | SIF | 7.65 | 14.6 |
| | 0.1N NaOH | 8.88 | 42.2 |

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A salt form of a compound of Formula (I): 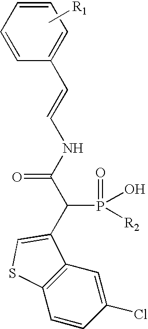
wherein
R₁ is one or two halogen substituents; and,
R₂ is C₁₋₄alkyl, C₁₋₄alkoxy, pivalyloxy-C₁₋₄alkoxy or hydroxy.

2. The salt form of claim 1, wherein
R₁ is two halogen substituents, wherein halogen is selected from fluoro or chloro; and,
R₂ is C₁₋₄alkyl, pivalyloxy-C₁₋₄alkoxy or hydroxy.

3. The salt form of claim 1, wherein the compound of claim 1 is selected from:
{(5-chloro-benzo[b]thiophen-3-yl)-[2-(3,4-difluoro-phenyl)-vinylcarbamoyl]-methyl}-methyl-phosphinic acid,
{(5-chloro-benzo[b]thiophen-3-yl)-[2-(3,4-difluoro-phenyl)-vinylcarbamoyl]-methyl}-phosphonic acid,
2,2-dimethyl-propionic acid {(5-chloro-benzo[b]thiophen-3-yl)-[2-(3,4-difluoro-phenyl)-vinylcarbamoyl]-methyl}-hydroxy-phosphinoyloxymethyl ester,
{(5-chloro-benzo[b]thiophen-3-yl)-[2-(3,5-dichloro-phenyl)-vinylcarbamoyl]-methyl}-methyl-phosphinic acid,
{(5-chloro-benzo[b]thiophen-3-yl)-[2-(3,5-dichloro-phenyl)-vinylcarbamoyl]-methyl}-phosphonic acid, and
2,2-dimethyl-propionic acid {(5-chloro-benzo[b]thiophen-3-yl)-[2-(3,5-dichloro-phenyl)-vinylcarbamoyl]-methyl}-hydroxy-phosphinoyloxymethyl ester.

4. The salt form of claim 1, wherein the salt form is selected from a benzathine, t-butylamine, magnesium, calcium, choline, cyclohexylamine, diethanolamine, ethylenediamine, L-lysine, NH₃, NH₄OH, N-methyl-D-glucamine, piperidine, potassium, procaine, quinine, sodium, triethanolamine, imidazole or tris(hydroxymethyl)methylamine salt.

5. The salt form of claim 4, wherein the salt form is selected from a magnesium, calcium, choline, N-methyl-D-glucamine, potassium, sodium or tris(hydroxymethyl)methylamine salt.

6. The salt form of claim 4, wherein the salt form is selected from a choline, N-methyl-D-glucamine or tris(hydroxymethyl)methylamine salt.

7. The salt form of claim 4, wherein the salt form is selected from a choline or tris(hydroxymethyl)methylamine salt.

8. The salt form of claim 4, wherein the salt form is a choline salt.

9. The salt form of claim 4, wherein the salt form is a mono-salt.

10. The salt form of claim 4, wherein the salt is a crystalline form.

11. The salt form of claim 1, wherein the salt form is present in an anhydrous or di-hydrate form.

12. The salt form of claim 1, wherein the salt form is present in an unsolvated form, a solvated form or an amorphous form.

13. The mono-choline salt of a compound selected from the group consisting of:
{(5-chloro-benzo[b]thiophen-3-yl)-[2-(3,4-difluoro-phenyl)-vinylcarbamoyl]-methyl}-methyl-phosphinic acid,
{(5-chloro-benzo[b]thiophen-3-yl)-[2-(3,4-difluoro-phenyl)-vinylcarbamoyl]-methyl}-phosphonic acid,
2,2-dimethyl-propionic acid {(5-chloro-benzo[b]thiophen-3-yl)-[2-(3,4-difluoro-phenyl)-vinylcarbamoyl]-methyl}-hydroxy-phosphinoyloxymethyl ester,
{(5-chloro-benzo[b]thiophen-3-yl)-[2-(3,5-dichloro-phenyl)-vinylcarbamoyl]-methyl}-methyl-phosphinic acid,
{(5-chloro-benzo[b]thiophen-3-yl)-[2-(3,5-dichloro-phenyl)-vinylcarbamoyl]-methyl}-phosphonic acid, and
2,2-dimethyl-propionic acid {(5-chloro-benzo[b]thiophen-3-yl)-[2-(3,5-dichloro-phenyl)-vinylcarbamoyl]-methyl}-hydroxy-phosphinoyloxymethyl ester.

14. A salt of claim 13 wherein the compound is {(5-chloro-benzo[b]thiophen-3-yl)-[2-(3,4-difluoro-phenyl)-vinylcarbamoyl]-methyl}-methyl-phosphinic acid.

15. A process for the preparation the salt form of claim 1, comprising the step of: 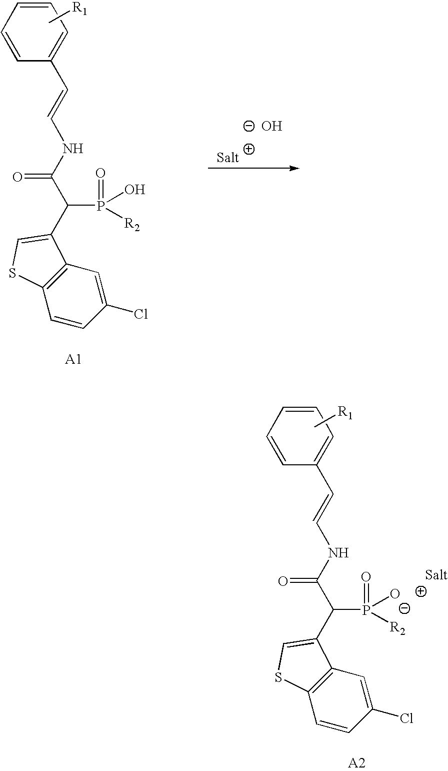
a. reacting a solution of one equivalent of a free acid form of a Compound A1 with a solution of an equivalent of a salt to provide a reaction mixture;
b. distilling the solvent from the reaction mixture to provide a residue;
c. solvating the residue obtained in step b. by adding a second solvent to provide a solution; and
d. precipitating a salt form of a Compound A2 from the solution.

16. The process of claim 15, wherein the free acid form of a Compound A1 is in a solvent selected from methanol, ethanol or mixtures thereof.

17. The process of claim 15, wherein the salt is in a polar organic solvent selected from methanol, ethanol, ethyl acetate, isopropyl alcohol or mixtures thereof.

18. The process of claim 15, wherein the second solvent is ethyl acetate.

19. The process of claim 15, wherein the equivalent of the salt used in step a. is in a range of from about 0.96 to about 1.16 molar equivalents, a range of from about 0.99 to about 1.13 molar equivalents, a range of from about 1.02 to about 1.1 molar equivalents, or a range of from about 1.04 to about 1.08 molar equivalents.

20. The process of claim 15, wherein the salt used in step a. is in the form of either a solid or a gas.

21. The process of claim 15, wherein the means of precipitating the salt form is selected from seeding the salt mixture in a solvent with crystals of the salt form, precipitating the salt by cooling, use of an antisolvent or by vapor diffusion crystallization with an antisolvent, forming the salt by rapid evaporation of the solvent from the salt mixture or slowly evaporating the solvent from the salt mixture.

22. The process of claim 21, wherein solvent:antisolvent pairs suitable for recovering the salt by crystallization with an anti-solvent are selected from methanol:acetone, water:acetone, ethanol:ethyl acetate and methanol:ethyl acetate.

23. The process of claim 21, wherein solvent:antisolvent pairs suitable for recovering the salt by vapor diffusion crystallization with an anti-solvent are selected from dichloromethane:acetone, dichloromethane:diethyl ether, dichloromethane:hexanes, dichloromethane:tetrahydrofuran and N,N-dimethylformamide:toluene.

24. The process of claim 15, wherein the precipitated salt forms are selected from:
{(5-chloro-benzo[b]thiophen-3-yl)-[2-(3,4-difluoro-phenyl)-vinylcarbamoyl]-methyl}-methyl-phosphinic acid choline,
{(5-chloro-benzo[b]thiophen-3-yl)-[2-(3,4-difluoro-phenyl)-vinylcarbamoyl]-methyl}-phosphonic acid choline,
2,2-dimethyl-propionic acid {(5-chloro-benzo[b]thiophen-3-yl)-[2-(3,4-difluoro-phenyl)-vinylcarbamoyl]-methyl}-hydroxy-phosphinoyloxymethyl ester choline,
{(5-chloro-benzo[b]thiophen-3-yl)-[2-(3,5-dichloro-phenyl)-vinylcarbamoyl]-methyl}-methyl-phosphinic acid choline,
{(5-chloro-benzo[b]thiophen-3-yl)-[2-(3,5-dichloro-phenyl)-vinylcarbamoyl]-methyl}-phosphonic acid choline, and
2,2-dimethyl-propionic acid {(5-chloro-benzo[b]thiophen-3-yl)-[2-(3,5-dichloro-phenyl)-vinylcarbamoyl]-methyl}-hydroxy-phosphinoyloxymethyl ester choline.

25. A crystalline choline salt of {(5-chloro-benzo[b]thiophen-3-yl)-[2-(3,4-difluoro-phenyl)-vinylcarbamoyl]-methyl}-methyl-phosphinic acid comprising the following X-ray diffraction peaks:

| °2θ | Å | % |
|---|---|---|
| 8.328 | 10.6177 | 71.32; |
| 10.069 | 8.7850 | 13.18; |
| 12.064 | 7.3367 | 12.10; |
| 14.202 | 6.2364 | 77.83; |
| 16.382 | 5.4110 | 34.77; |
| 18.599 | 4.7708 | 10.95; |
| 19.206 | 4.6213 | 100.00; |
| 19.845 | 4.4740 | 53.54; |
| 19.955 | 4.4496 | 57.38; |
| 20.181 | 4.4002 | 60.89; |
| 20.584 | 4.3151 | 40.36; |
| 21.101 | 4.2104 | 11.74; |
| 21.300 | 4.1715 | 13.23; |
| 22.089 | 4.0243 | 83.79; |
| 22.833 | 3.8949 | 50.10; |
| 24.049 | 3.7006 | 26.09; |
| 25.257 | 3.5262 | 14.17; |
| 25.894 | 3.4409 | 11.04; |
| 26.713 | 3.3373 | 30.15; |
| 28.522 | 3.1296 | 36.20; |
| 29.733 | 3.0048 | 18.27; |
| 30.521 | 2.9266 | 19.90; and |
| 31.579 | 2.8333 | 14.15. |
